(19) United States
(12) Patent Application Publication
(10) Pub. No.: US 2021/0322735 A1
(43) Pub. Date: Oct. 21, 2021

(54) MEDICAL DRAINAGE DEVICE AND USES THEREOF

(71) Applicant: Virginia Commonwealth University, Richmond, VA (US)

(72) Inventors: Stephanie Goldberg, Richmond, VA (US); Jonathan H. DeAntonio, Henrico, VA (US); Hilton Bennett, Richmond, VA (US)

(21) Appl. No.: 17/234,001

(22) Filed: Apr. 19, 2021

Related U.S. Application Data
(60) Provisional application No. 63/012,439, filed on Apr. 20, 2020.

(51) Int. Cl. A61M 27/00

(52) U.S. Cl. CPC A61M 27/002; A61M 2027/004

(57) ABSTRACT

A medical drainage device for draining air or fluid from a body cavity is described for its use in treating a subject suffering from pneumothorax and/or pleural effusion. The device comprises a tube or a catheter-over-needle for receiving air or fluid from the body cavity and one or more fixation systems for preventing inadvertent dislodging of the needle and/or catheter during the draining procedure. A woven semi-flexible catheter coated with antithrombic agents is used for reducing kinks and clogs. The device may be used for draining air and/or fluid from a plurality of types of body cavities and is particularly suitable for uses in military and/or emergency settings.

### CROSS-REFERENCE TO RELATED APPLICATIONS

This application claims priority to U.S. Provisional Application 63/012,439 filed on Apr. 20, 2020. The complete content thereof is herein incorporated by reference.

### FIELD OF THE INVENTION

The disclosure generally pertains to a drainage device, more specifically a thoracic drainage device comprising a fixation system and a method of using such device for treating a subject suffering from pneumothorax and/or pleural effusion.

### BACKGROUND

Penetrating chest trauma is a critical medical emergency which must be treated with urgency as one can perish in as little as two seconds or up to two days if left untreated. More than 50% of patients suffering from chest trauma concomitantly develop pneumothorax which is a condition representing a collection of air in the pleural space. Generally, pressure around the lungs is lower than atmospheric pressure outside the body. When the negative pressure in the intrapleural space is disrupted (e.g., by chest trauma such as stabbing, diseases implicated with fluid or air build-up, etc.), the lung will partially or completely collapse. Pleural effusion is another condition which may be developed from chest trauma, in which the condition is defined as an excess of fluid accumulation in the pleural space. Pneumonia and cancer may cause a pleural effusion or an excess amount of fluid or blood to build up in the cavity around the lungs. Similarly, the collection of blood in the pleural space results in a condition commonly referred to as hemothorax, in which case the lung not only collapses but the air and fluid within the cavity builds up pressure to cause blockage of veinous blood flow back to the heart, which may result in cardiac arrest and death unless treated immediately. Other types of pleural effusions include chylothorax (collection of lymph chyle due to disruption of the thoracic duct) and empyema (collection of pus). Pneumothorax is the second most preventable life-threatening condition facing the U.S. troops and accounts for one-quarter of trauma casualties. In this case, quickly relieving the pressure in the pleural space is critical for patient survival. Two of the most used medical procedures for air or fluid removal are thoracentesis (fluid removal from the pleural cavity) and paracentesis (fluid removal from the abdominal cavity). In the course of such medical treatment procedures, air and body fluids are withdrawn from a patient.

The use of a thoracic drainage device containing catheters (e.g., chest drain, chest tube, intercostal drain, tube thoracostomy, wide-bore needles) for removing air (for pneumothorax) or fluid (e.g., blood, effusion, chyle or pus) from the chest cavity of a patient is known in the art. The most commonly used for treatment is a chest tube thoracostomy, which is a medical procedure performed to drain fluid, blood or air from the space around the lungs. Currently, various types of catheters are being used for treating pneumothorax, tension pneumothorax, large pleural effusions, chylothorax, hemothorax and empyema. Such catheters are also frequently used in the post-operative setting following a thoracotomy, esophagectomy, video-assisted thoracoscopy surgery (VATS) and cardiac surgeries. Typically, the size of the catheter or chest tube inserted into a pleural cavity may vary depending on the degree of severity of diagnosed pneumothorax. Generally, 15-30% of collapsed lung may use a needle while any percentage larger than 30% may require a tube.

Traditional treatment procedure utilizes a chest tube and directly inserts the chest tube into the pleural space for draining accumulated materials. In order to prevent inadvertent removal and/or movement of the device, once the thoracic catheter (i.e., chest tube) is in place, the catheter is taped or otherwise attached (i.e., adhere the device to a patient body by glue, tape or sutured) to the patient's skin. U.S. Pat. No. 9,616,203 to Donaldson and U.S. Pat. No. 9,072,823 to Hopman describe such adhesive mechanism and as well as a “Veress” or “Veress-type” needle-containing portable thoracic draining device that may be used in emergencies. The use of needle or needle thoracostomy is especially useful in the out-of-hospital emergencies and/or military environment. Some devices are further connected to other accessories and/or a suction source (e.g., negative 20 cm of water pressure) or on a water seal for improved drainage. Examples of the thoracic drainage systems of similar kind are described in U.S. Pat. No. 8,257,339 to Rosado and U.S. Pat. No. 9,839,726 to Ehlert.

Such devices or strategies, however, do not provide sufficient fixative mechanism for the device and do not resolve the complications of the catheter forming kinks and clogs. Although the Veress needle type with a blunt drainage needle attached to a retractile sharp introducer may reduce the likelihood of the sharp needle damaging internal tissue during procedure, however, the blunt-end needle itself does not improve the safety of the procedure especially when the inserted needle, regardless of the shape of the needle-tip, is unstable during the procedure. Again, the known strategies to secure this kind of device are to adhere the device to a patient by tape, glue or suture. With strategies that incorporate an adhesive base that fixates the inserted needle in place, the device may be insufficient to be used in uncontrolled emergency situations (e.g., military uses). Under these situations, the chest area around the insertion point would most likely be difficult to clean, lowering the adhesive's effectiveness. In addition, the size and complexity of the previously known devices make them ill suited for their uses in emergency settings. Clogging inside of the device by thrombus formation or debris, if not controlled properly, may cause additional major subsequent complications such as subcutaneous emphysema (backpressure created by undrained air) and malfunctioning of the device. Subsequently, other complications such as hemorrhage, infection, and injuries to underlying organs (e.g., heart, liver, or spleen, etc.) may follow from the use of clogged tube and accidental puncture due to the device's instability.

Thus, there is a need in the art for improved thoracic drainage device and more specifically for such device in emergency usage that does not have the current high failure rate. Currently, the needle decompression method, under emergency, non-clinical, military settings in particular, experience over 58% failure rate. Instability of the thoracic drainage device before, during and after the drainage procedure is a great concern. A thoracic drainage device that can provide stability during the medical procedure in the most rapidly effective, minimally invasive and cost-effective manner is needed.

### SUMMARY OF THE INVENTION

The portable drainage device, according to some embodiments of the disclosure, is a medical drainage device that contains an integrated system that allows for fixation or stabilization of the device on a subject during the drainage procedure. The device may comprise one or more inflatable balloons as a fixative system thus overcoming and substantially alleviating the deficiencies in the prior art by providing a method to rapidly perform air or fluid removal from a body cavity especially in emergency settings with a minimally invasive approach. The device may also comprise antithrombin treated portions and a woven catheter, to lessen the risk of clogging/kinking in the device during the treatment procedure.

A primary object of the invention is to provide a medical drainage device having one or more fixation systems and a semi-rigid tube. In some embodiments, the fixation system may be one or more balloons that wrap around the tube and/or be placed on the outer surface of the tube as not to interfere the air and/or fluid drainage flow occurring in the lumen of the tube. In some embodiments, the balloons are inflated by blowing air through one or more inflation channels that extend and run on the longitudinal axis, either inside or outside the tube, with a port on the outer surface of the tube. In other embodiments, a plurality of other securing fixation systems may also be included in the device, with or without the inflatable balloons, such as flanges, balloons, external foam bumpers adjusted to skin level, rubber liners, for preventing balloons or any listed securing systems from sliding down or up the tube. In some embodiments, the device also comprises a retractable needle system as well as a stylet with a spring-loaded protective guide over the needle tip so that the risk of puncturing the lung or any other underlaying internal organs is minimized. In some embodiments, the tube comprises one or more one-way valves to prevent the drained air or fluid flowing back into the body cavity. In some embodiments, the tube is a catheter. In some embodiments, the catheter is woven or any flexible material capable of forming a semi-rigid hollow tubing. The catheter may have one or more luer locks and/or syringe adaptors on the proximal end, providing air-sealed connections to additional components or accessories (e.g., syringes, inflator bulb, catheter extension, pump, aspirator, etc.).

Another aspect of the disclosure provides a method of using the medical drainage device for draining air or fluid from a subject in need thereof using a device according to the disclosure by following the steps of: inserting a tube to a body cavity of the subject; inflating one or more balloons to an appropriate size, wherein the size of the inflated balloon is sufficient to hold the inserted tube in position; draining out air or fluid from the body cavity through the catheter for an appropriate time; deflating the balloons; and removing the catheter. In some embodiments, the body cavity may be any types of cavities (anatomically present or newly formed) inside the body. The balloons and additional securing or fixative systems may be placed inside and/or outside the subject's body. In some embodiments, the method further comprises a step of making an appropriate size of an incision to place the tube (e.g., needle and catheter as a single unit) into a body cavity of the subject.

Another aspect of the disclosure provides a method of using the medical drainage device to treat a patient undergoing pneumothorax and/or pleural effusion. In some embodiments, a needle tip may be inserted through the chest wall to extend into a pleural cavity of the subject. Upon the insertion of the needle, one or more balloons that are attached to a wall of the catheter will be inflated to an appropriate size, thereby securing the position of the inserted needle and/or the catheter from moving. In some embodiments, the needle may be retracted upon insertion and/or after fixation in the pleural cavity by activating a retractable needle system of the device. In some embodiments, the method further comprises a step of making clinical diagnosis by observing mechanism of injury, respiratory distress, hypoxia, absent breath sounds, and/or hypotension.

Additional features and advantages of the present invention will be set forth in the description of disclosure that follows, and in part will be apparent from the description of may be learned by practice of the disclosure. The disclosure will be realized and attained by the compositions and methods particularly pointed out in the written description and claims hereof.

### BRIEF DESCRIPTION OF THE DRAWINGS

FIG. 1A is a schematic of an exemplary drainage device according to some embodiments of the disclosure.

FIG. 1B is an exemplary needle component of the drainage device of FIG. 1A.

FIG. 2A is a side view of an exemplary drainage device according to some embodiments of the disclosure.

FIG. 2B is another side view of the exemplary drainage device of FIG. 2A.

FIG. 3A is a perspective view of a catheter of the drainage device according to some embodiments of the disclosure.

FIG. 3B is a side view of another exemplary drainage device showing another means of fixation system using an adhesive tape.

FIG. 4A is a perspective view of a drainage device with two luer locks according to some embodiments of the disclosure.

FIG. 4B is a side view of another exemplary drainage device showing a balloon and an adhesive tape as an exemplary stabilizing system.

FIG. 5 is a perspective view of an exemplary drainage device with four balloons as an exemplary fixation system according to some embodiments of the disclosure.

FIG. 6 is a cross-sectional view of a drainage device showing an inflated balloon around the catheter stabilizing the device in a body cavity.

### DETAILED DESCRIPTION

Embodiments of the disclosure provide a medical drainage device for use in draining air or fluid from a body cavity of a subject in need thereof. In particular, the disclosure provides a mechanism that allows stabilizing the device during a drainage procedure. In some preferred embodiments, the device comprises one or more inflatable balloons as a fixation mechanism to overcome and to substantially alleviate the deficiencies in the prior art by providing a method to rapidly perform air or fluid removal from a body cavity with a minimally invasive approach. The device is portable, disposable, simple to use and can be packaged as a preassembled and/or constructible kit so that the device may be used not only in hospitals and clinics, but also may be appropriate for outdoor, emergency or military uses.

A medical drainage device, in some embodiments, comprises a plurality of types of tubes, in which the term “tube” is defined herein as a hollow, cylindrically shaped material or combinations of such materials forming a portion of the device. The tube has a proximal end (distant from the body cavity) and a distal end (toward the body cavity) that opens to accept and transfer air and fluid from the body cavity. The tube may be a catheter, a needle, a hose, a pipe, a conduit, a main, a duct, a line, a channel, a pipeline, a drain, a siphon, a cannula or any hollow cylindrical shaped object.

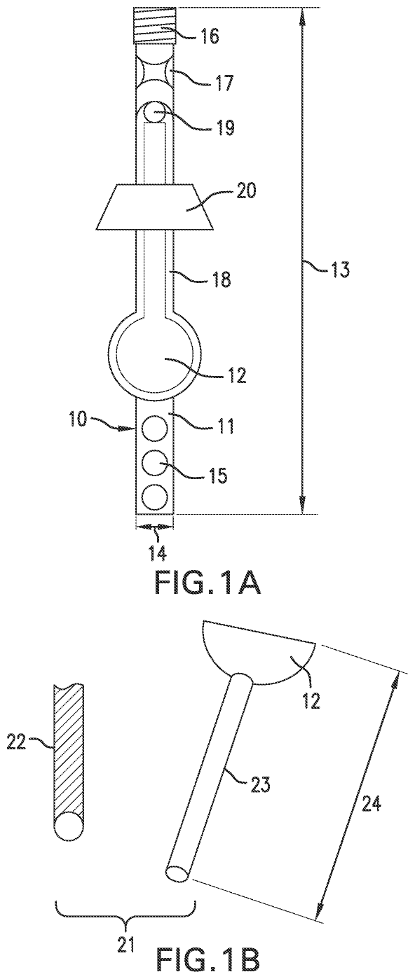

With reference to FIG. 1A, in some embodiments, a drainage device 10 comprises a catheter 11 and an inflatable balloon 12 that wraps around a distal portion of the catheter 11. In some embodiments, the device has a total length 13 (i.e., length of the catheter) of about 8-22 cm, preferably 9-20 cm, or more preferably 10-18 cm. In some embodiments, the device has a diameter 14 (i.e., diameter of the catheter) of about 11-17 Fr (3.6-5.7 mm), preferably 12-16 Fr (4-5.3 mm) or more preferably 13-15 Fr (4.3-5 mm). The catheter 11 has at least 2 or more, preferably 3 or more, more preferably 4 or more asymmetric holes 15 on the distal end. The holes are of sufficient size and quantity to allow for passage of fluid, thrombus and debris that might need to be removed from the chest cavity. Further, in some embodiments, the inner lumen and/or outer surface of the catheter 11 may be impregnated with antifibrinolytic agents, antithrombin agents, anticoagulant agents, antimicrobial agents or any combinations thereof. Exemplary antithrombin agents may include, but are not limited to, heparin, heparin sulfate, hirudin, chondroitin sulfate, dermatan sulfate, keratin sulfate, lytic agents, including urokinase and streptokinase, their homologs, analogs, fragments, derivatives and pharmaceutical salts thereof.

In some embodiments, the catheter 11 is made of a material or in combinations of materials that create reasonable flexibility, such as polymeric, polycarbonate, olyvinyl chloride, poly esters, polyethylenes, polyamides, silicone and polyisoprenes, metals and ceramics. Other non-limiting polymer classes that may be suitable for formation of the catheter include Styrene Butadiene Styrene (SBS) polymers such as Kraton®, Dynaflex® and Santoprene®, Vinyl compounds, silicone rubber materials such as poly-dimethylsiloxane, thermoplastic polyester elastomers (TPEs), and polyether block amides such as Pebax®. In some preferred embodiments, a woven catheter is used to lessen the risk of clogging and/or kinking in the device during the drainage procedure. In some embodiments, the catheter is a non-kinking catheter. For example, the catheter may comprise a combination of a woven material and a wire lining material. In some embodiments, the catheter comprises a semi-rigid cavity or body, in line or parallel with the catheter's access hole. In catheters having multiple holes, one or more of the holes (including the central access hole) may be formed from or filled with a semi-rigid material while the remaining portions of the catheter are more flexible. In some embodiments, each of the cathether holes are formed from or filled with a semi-rigid material or wire.

The proximal portion of the catheter may include a luer lock 16, syringe adaptor or any adaptor that may be suitable for connecting various additional appliances (e.g., pump, aspirator, syringe, bulb, etc.). In some embodiments, one or more one-way valves 17 may be incorporated into the catheter, preferably on a proximal portion of the catheter. The one-way valve comprises an inlet end that opens for receiving air or fluid to be drained and an outlet end that is in a normally closed position and is adaptable to partial opening to permit drainage of the material received from the inlet. Exemplary one-way valve in a medical device is described in U.S. Pat. No. 7,533,696 to Paul, Jr., herein incorporated by reference.

In some embodiments, a fixation system is attached to an outer wall of the catheter. A preferred fixation system, in some embodiments, is one or more balloons. As shown in FIG. 1A, a balloon 12 is fixed by winding around the distal portion of the catheter so that the balloon and some portion of the catheter may be inserted to a body cavity as a single unit. The term “balloon” herein refers to a flexible bag made of any types of poreless materials that can be inflated with any types of materials including gas (such as helium, hydrogen, nitrous oxide, oxygen, air, smoke, etc.), fluid (water, oil, etc.) or granular media to provide a volume. A plurality types of materials may be used to manufacture the balloons described herein, such as rubber, latex, polyisoprene, nylon fabric, silicone, urethane, mylar or other biocompatible elastomeric materials that are well known in the art. The balloon should have sufficient impermeability properties to substantially prevent inflation gas or fluid from permeating through a wall of the balloon. In some embodiments, the balloon may be made with materials that are inadequate to provide desirable impermeability or inflatability but later coated or sealed with another appropriate material or combinations or materials known in the art (e.g., greased or coated porous silicone balloon). Different shapes of balloon may be pre-determined and manufactured depending on the size and shape of body cavity or insertion site. Exemplary shapes of a balloon may include, but are not limited to, donut, cone, collar, sphere, triangular pyramid, pillow, umbrella, cylinder, rectangular prism, triangular prism, hexagonal prism or any other contoured shapes. Additionally, the inflated form of balloon may take different shapes such as toroid, disc, fluted or any combinations of shapes that may be pre-molded.

In other embodiments, inflation and deflation of balloon are controlled through an inflator channel 18 that is connected to each balloon and embedded along a partial or full longitudinal length of the inner wall of the catheter 11. When multiple balloons are present in the device, the volume of each balloon may be controlled separately. A controlled volume inflation-deflation device may also be attached with increments of controlled, selected, and/or equal volumes of air to control the outer diameter of the one or more balloons to predetermined or adjusted outer diameters. The outer diameter of the balloon may be controlled by inflating and deflating with selected or controlled volumes of relatively incompressible liquids and solutions, which may vary depending on the intended usage. The balloon inflation channel 18 and the channel port 19 are partially or completely housed within the inner lumen or embedded in the wall of the catheter. However, there is no communication between the drainage lumen and balloon inflation channel. In some embodiments, one or more inflation channels may be used. In these embodiments, each inflating channel has a port at a proximal portion of the catheter. The inflation port 19 may be positioned just below the luer lock 16 to allow majority of the catheter to insert into the chest of a subject. An exemplary balloon inflation system may include a syringe or a bulb, a volume of balloon inflation fluid such as sterile saline or air and a valve or a stopcock. When in deflated state, the balloon 12 is sized and configured to fit snugly and lie flat around the catheter 11. One or more sliding bumpers 20, for the purpose of preventing the inflated balloon 12 to distally slide on the catheter, may also be included in the device. The bumper 20, in these embodiments, is placed around the outer surface of the catheter 11. In some embodiments, the inner portion of the bumper 20 is lined with materials that are slide-resistant (e.g., rubber) that fits snugly around the outer wall of the catheter 11. After inserting the catheter into a body cavity, the later inflated balloon may be held against the surface of the subject's internal body wall while the exterior wall surface may be flush with an additional fixation system, in this case, bumper 20. By clamping the device onto the subject's body with two or more fixation systems, the device will remain stable throughout the draining procedure. In some embodiments, the position of bumper 20 is easily adjustable by frictionally engaging or sliding the bumper on catheter. In some embodiments, a balloon is to be inflated outside the subject's body as a means to stabilize the inserted catheter. In these embodiments, bumper 20 is moved to a position so that the bumper is flush with the inflated balloon 12 and thus supporting and fixating the device in place. Unlimited numbers of balloons and bumpers may be used to secure the inserted tube (i.e., catheter) against the skin so that the device is stabilized and immobile during the procedure. In most preferred embodiments, the balloon is larger than the incision or insertion hole. The inflated balloon has a diameter of about 0.5-4 cm, preferably 0.8-3 cm, more preferably 1-2.5 cm. In some embodiments, the balloon may have a size smaller than the incision site especially when one or more balloons are used in combination. In this instance, the one or more balloons together have a size larger than the incision or insertion hole when inflated.

As shown in FIG. 1A-B, in some embodiments, the catheter 11 comprises a needle or cannula 21, which may be a Veress-type needle or a safety decompression needle, where the needle may further include a stylet 22 or a blunt sub-needle in a retractile sharp introducer needle 23. Any protective, atraumatic needle tips (e.g., stylet) with a spring or any other needle retractable system known in the art may be used to prevent the needle from puncturing neighboring or underlying organs in the body upon insertion. In these embodiments, a plurality of needle retracting systems known in the art may be used, however, preferably a controller of the retractable system is disposed on the proximal side of the device. For example, a retracting pen mechanism where a hollow outer catheter is ground obliquely to a sharp point at the distal end of the catheter, and the proximal end is mounted in a pen-like housing, may be used. A “click pen” mechanism, which allows the sharp point of the device to be exposed or obscured through the user's action, may also be utilized and the detailed mechanism is described in U.S. Pat. No. 9,072,823 to Hopman, herein incorporated as reference. In some embodiments, the device present in this disclosure may be manufactured to enable one-step operation. In some embodiments, the needle has a length 24 of about 6-24 cm, preferably 7-23 cm, more preferably 8-22 cm and with an external diameter of 0.5-4 cm, preferably 0.8-3 cm, more preferably 1-2.5 cm. Moreover, the needle may have a size of about 14-20 G, preferably 14-18 G, more preferably 14-16 G. In some embodiments, a safety needle retractive system may be incorporated.

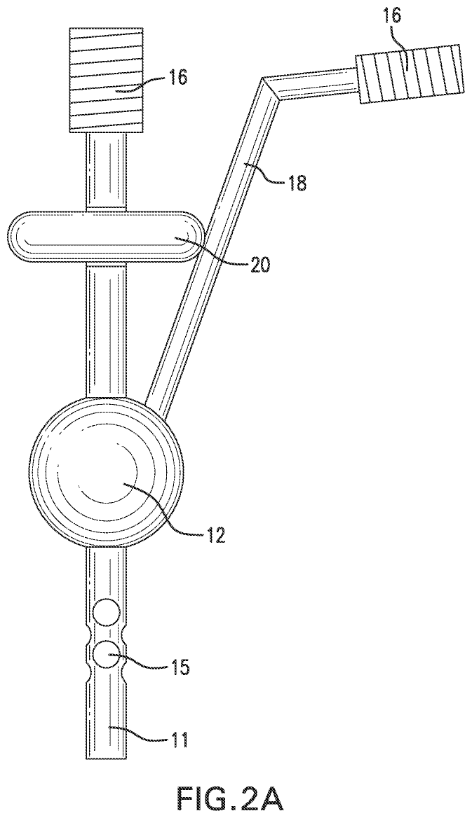

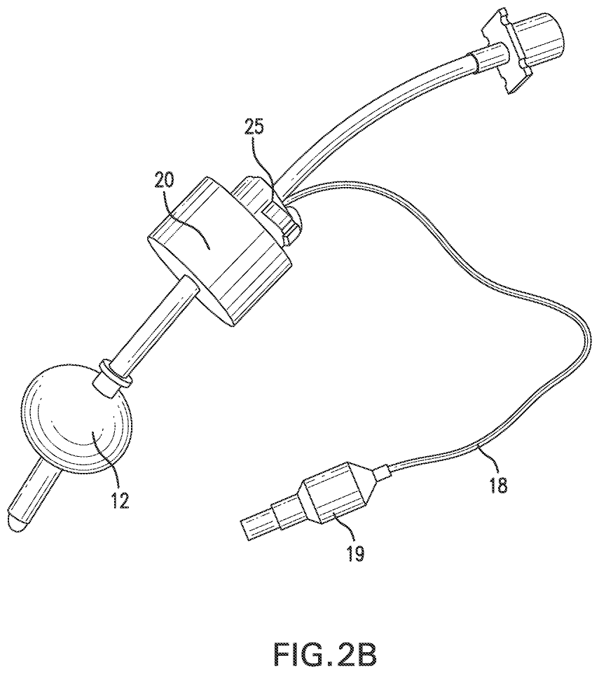

With reference to FIG. 2A-B, in some embodiments, the balloon inflation channel 18 is partially or completely housed on the outer surface of the catheter. Of note, there is no communication between the drainage lumen and balloon inflation channel. In these embodiments, inflation port 19 may or may not be attached to the catheter or any portion of the device. In some embodiments, one or more inflation channels 18 may be used. In such cases, a clip or a holder 25 may be used to collect, bind and attach the channels to the catheter as well as removing the risk of introducing kinks or folds in the channels. In some embodiments, one or more clips 25 may be placed below and/or above the sliding bumper 20 (e.g., foam, rubber, flange, balloon or any material that may fasten the clamps). In other embodiments, one or more clips 25 may be adjacently placed with respect to the fixation system 12 and/or 20 toward proximal end of the catheter. Other embodiments of the drainage system may alternatively include different types of removable constrictions around the catheter and the air channels for the balloons, such as a snap-fit belt, a removable tie or wrap, a band clamp, an o-ring, a connector, a rubber band etc. Such constriction may provide a locking means to substantially prevent or minimize the catheter and the balloon inflator channels from dissociating, particularly during insertion of the device to a body cavity.

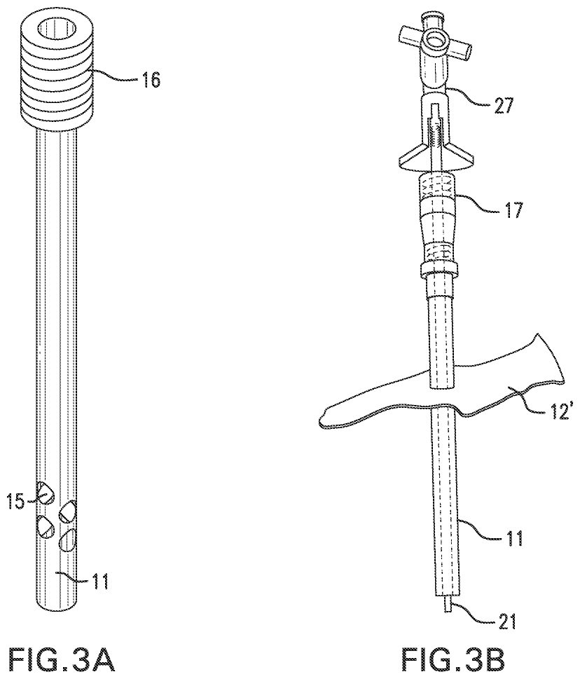

With reference to FIG. 3A-B, in some embodiments, a drainage device comprises a catheter 11 with one or more holes 15 on a distal portion for draining air or fluid without clogging; a one-way valve 17 attached on the proximal end of the catheter; a needle retracting system 27; and one or more adhesive materials 12′ to attach the device on the skin of a subject. In some embodiments, the catheter 11 may be shaped in a way to incorporate an intrinsic fixation mechanism, such as the catheter shaped to form radial protrusions, a groove, a bent or a wave, which may be manufactured by utilizing soft deformable plastic or any other appropriate materials known in the art. Moreover, the catheter 11 may also be formed in varying shapes including a straight elongated shape, a flared shape, a curved shape, a coiled shape, a pig-tail shape, a J-shape and the like. In preferred embodiments, the catheter is a polyurethane woven. In some embodiments, the catheter is in any yarn patterns such as a braided pattern, a woven pattern, a knitted pattern, a filament wound pattern or combinations thereof. The catheter may include additional internal coatings, such as Teflon, tethered-liquid perfluorocarbon, silver ionic, silver nanoparticles, copper, copper alloy, organosilanes, gold, titanium dioxide, and carbon nanotubes for preventing clot or blockage and reducing the risk of infection. Further, all parts of the device (e.g., needle, catheter, valve, channel, etc.) may be coated with therapeutic agents or metals that are listed above. In some embodiments, the catheter may be large enough to accommodate additional devices, such as imaging device, camera, thoracoscopy device, etc. In some embodiments, the catheter may be thin and may not comprise a needle. In some embodiments, one or more balloons may replace the adhesive materials 12′ shown in FIG. 3. In some embodiments, the adhesive 12′ can be used in combination with balloons. Alternatively, the attached adhesive setting is separated or removed from the balloons prior to normal operation if the adhesive interferes with the inflation of the balloons. The balloons may be tested or visually inspected before and/or during use of a drainage system. Such inspection can bring to light damaged balloons (e.g., non-inflatable or tear) improper operation, insufficient inflation levels, etc.

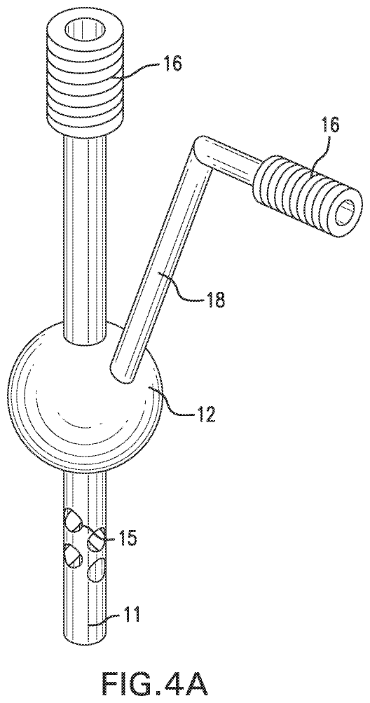

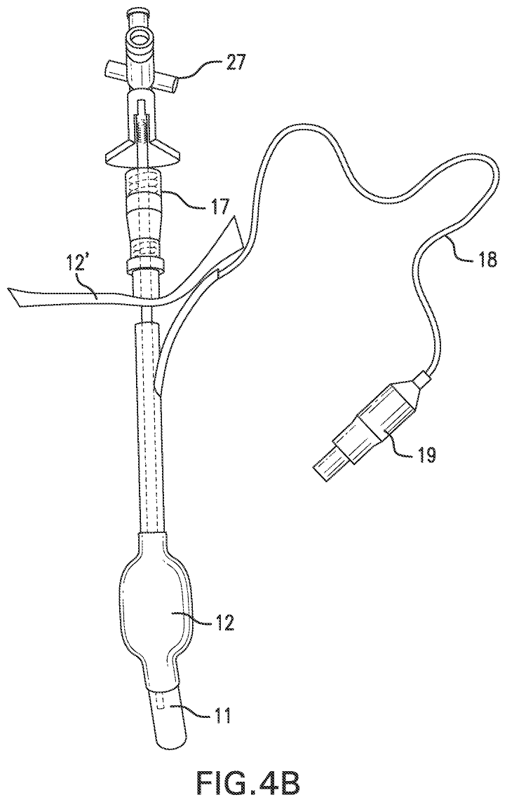

With reference to FIG. 4A-B, the drainage device may comprise multiple fixation system components (i.e., bumper shaped catheter, one or more balloons and/or adhesive materials). In some embodiments, the adhesive 12′ may be medically appropriate tape (e.g., “3M” Medical Foam Tape, MEDIFIX® 4005, etc.) or elastogel, protective gel, tape embedded with protective sheet, porous sheet, non-woven adhesive, etc. In some embodiments, the device may have, in addition to a one-way valve 17, a controller with openings for connecting a suction and/or an extension. The medical drainage device according to the present invention includes one or more one-way valves 17 that are applicable even in varying ranges of the air pressure in the cavity (e.g., very low positive pressure relative to the atmospheric pressure) so that the backflow of the drained air and/or fluid does not occur. In some embodiments, the inflation channel has a luer taper 16 at the distal end for making leak-free connections between a male-taper fitting and its mating female part. In some embodiments, one or more leur lock systems 16 are incorporated in the distal portions of the device.

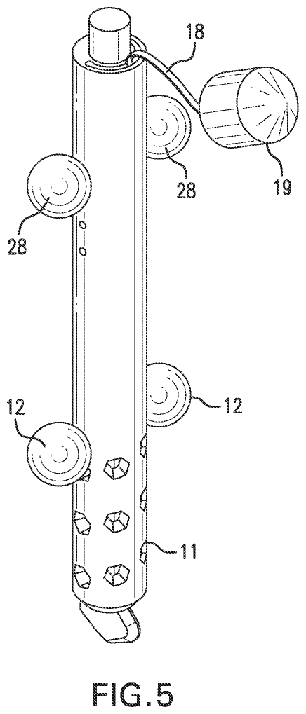

In some embodiments, as shown in FIG. 5, one or more balloons 12 are attached on the outer wall of the catheter. Each balloon may be placed inside or outside of the body of a subject. As such, one or more inflatable channels 18 for balloons are attached to the catheter. The balloons that are placed outside of the subject's body may be reversibly or non-reversibly expanded. Some of the balloons may be inserted into the body with catheter as a single unit and the balloon may serve to push tissue away from the inserted needle or catheter for placement into the body cavity. In some embodiments, the balloons may be used to dilate the punctured or incised opening in the tracheal wall. One or more valves or stopcocks on the balloon inflation system may be used to maintain the balloon in the inflated or deflated configuration. As shown in FIG. 5, one or more balloons may be permanently affixed to the catheter. Alternatively, one or more balloons 12 and 28 may be adjustable, removable or detachable depending on the intended use of the device. At the same time, the inflation channels of the balloons may be placed in the lumen of the catheter. In some embodiments, the inflating channels may be attached outer surface of the catheter and completely or partially detachable. In some embodiments, a device may have multiple balloons affixed to the catheter and one or more balloons 28 may be inflated outside of the subject's body while one or more balloons 12 are concomitantly inflated intracorporeally upon insertion of the catheter. In some embodiments, the inflation of one or more balloons 28 that are placed outside the body occurs after the inflation of intracorporeal one or more balloons 12 so that the outer balloons, upon inflation, may provide a seal for the insertion site and/or fix the inserted catheter from dislodging. Such locking mechanism will keep the device from moving once the device is in the desired position.

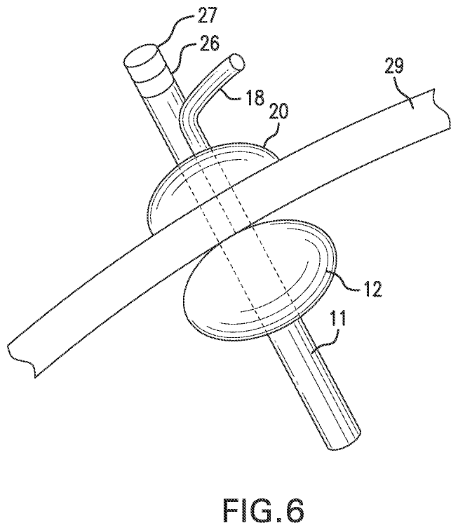

With reference to FIG. 6, a device of the disclosure is used in a method for draining air or fluid from a subject using the device by following the steps comprising: i) making a small incision on the subject's skin for the tube to advance through the skin. ii) inserting a tube of the device to a body cavity through the incision; iii) inflating one or more balloons 12 of the device to an appropriate size and confirm the size of the inflated balloon is sufficient to hold the inserted tube in position; iv) opening a one-way valve 17 to operate; v) draining out the air or fluid from the body cavity through the inserted tube 11 for an appropriate time; vi) deflating the balloons 12; and vii) removing the tube 11. In some embodiments, a step of activating one or more adhesives 20 and/or other types of fixation systems is provided. A device of this disclosure may be used to drain air or fluid from body cavities of including, but not limited to, cavum peritoneal, atrium of heart, pleura internal layer of lung, gall-bladder, abdominal cavity, wound, or any other anatomically present or newly formed body cavities. A device of this disclosure is utilized to drain air or fluid from a subject's body cavity and the “subject” herein refers to a mammal, preferably human patient. The tube, in some embodiments, may be a needle or a catheter-over-needle. In addition, some embodiments of the present disclosure provide a step of determining the size of tubing (e.g., body tubes or needles) via diagnostic observation by clinicians to minimize pain and the risk of infection. To minimize the infection of the insertion site, a step of cleaning and/or disinfecting the skin 29 or the body of a subject may be included.

Preferably, the needle or tube of the device is connected as a single unit and the majority of the catheter, for example at least ½ of the length, more preferably ⅔ of the length, is inserted into the body of a subject. In some embodiments, the inflating channel may be attached to the outer wall of the catheter and thus a portion of the channel also enters the body. The time period for use of the drainage device can vary from minutes to hours to days, e.g., from 12 hours up to or even exceeding 30 days. The desired time period for use of the drainage device will vary based on the nature of the disease, the medical professional's recommendations, the heating rate of the patient, etc. There is no time limit beyond which use of the medical drainage device is no longer beneficial.

In preferred embodiments, a device of the disclosure is used in a method for treating pneumothorax by draining air or fluid from a subject using the device by following the steps comprising: i) cleaning or sanitizing a chest skin of the subject prior to a needle insertion ii) inserting a needle through a chest wall so that the needle tip extends into a pleural cavity of the subject; iii) inflating one or more balloons of the device to an appropriate size, wherein the size of the inflated balloon is sufficient to place the inserted needle and the attached catheter in a fixed position; iv) retracting the needle by activating a retractive needle system; v) opening one or more one-way valves to operation position vi) draining out the air from the pleural cavity through the catheter for an appropriate amount of time; vii) deflating the balloons; and viii) removing the catheter. In some embodiments, the incision may be made with appropriately preparing and anesthetizing a subject. The size of the incision will be determined depending on the volume of the drained materials as well as the diameter of the catheter used. In addition, some embodiments, one or more balloons may be fixated on the distal end of the catheter. In other embodiments, the balloons may be replaced with adhesives or any other forms of securing measures of the device (e.g., foam, block, bumpers, etc.). Any other forms of securing methods including flange, balloon, balloon and bumper and/or a series of small balloons to help maintain the catheter in the chest wall are used. In some embodiments, the device will be placed in same anatomical position as for a needle decompression thoracostomy (i.e., between 2^{nd} and 3^{rd} intercoastal spaces, above the rib). The catheter and stylet-type needle are held perpendicular to the skin and with steady and deliberate pressure, the combined catheter and stylet as a single unit may be inserted into the skin, through the chest way and into pleural space. In some embodiments, the needle “click pen” retractile component may be included and the protective center of the needle will re-extend once in the pleural space with an audible click. The practitioner will note the measured depth of the catheter at this time and the stylet is removed. The catheter is generally inserted approximately 0.5-5 cm, preferably 1-4.5 cm, more preferably 2-4 cm more.

All or some of the components of the device is made portable, small enough for a medical use outside of the hospital or clinical settings. Although the device itself does not require a pump assembly, however, it may be assembled with a pump or any other devices known in the art that are portable and suitable for assisting drainage of air or fluid material from body cavity. In some embodiments, portions or entirety of the device may be disposable in order to limit either possible infections or contaminations. Alternatively, some portions of the device may be kept for limited multiple uses if some single-use parts are appropriately attached and disposed after each use. Depending on the use of the device, different sensors or diagnostic accessories (e.g., air flow sensor, diagnostic probe, etc.) known in the art may be attached to the device, for example, via tubing. Additionally, the device may include a pressure sensor, a microcontroller, a screen or the like that displays the changes of pressure in the cavity (e.g., before and after restabilization of pressure in the cavity). For example, the attached sensor or screen may be placed in line with the catheter via a plurality of types of arrangements, such as “T” or “Y” configurations. In some embodiments, the device includes an audio, haptic or visual response in relation to stabilization or destabilization of the pleural cavity and/or an in between condition such as a “yellow light” visual response. The response may be displayed on a screen and/or processed through an attached circuit board. In preferred embodiments, a device of this disclosure may be used for traumatic and spontaneous pneumothorax, in particular, during field decompression of pneumothorax by EMS or military medics and/or decompression by providers not comfortable or appropriate trained to place tube thoracostomies (i.e., chest tube). In some embodiments, the method further comprises a step of making clinical diagnosis by appropriate medical professionals observing mechanism of injury, respiratory distress, hypoxia, absent breath sounds, and hypotension. The device may also be used in non-emergency settings for the treatment of spontaneous pneumothorax as a less invasive measure than a chest tube placement.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided it is understood that each intervening value, to the tenth of the unit of the lower limit unless the context clearly dictates otherwise, between the upper and lower limit of that range and any other stated or intervening value in that state range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

What is claimed is:

1. A medical drainage device comprising:
i) an elongated wire-lined or woven catheter having a proximal end and a distal end;
ii) a puncturing system at the distal end of the catheter for penetrating a body cavity, wherein the puncturing system comprises an outer cannula with a beveled tip and an inner cannula with a blunt tip, wherein the inner cannula is extendable and retractable; and
iii) one or more fixation systems attached to an outer surface of the catheter, wherein the one or more fixation systems are an appropriate size for stabilizing and halting movement of the device during a drainage procedure upon insertion of the catheter in the body cavity.

2. The drainage device of claim 1, wherein the catheter has a length of 10-18 cm.

3. The drainage device of claim 1, wherein the puncturing system has a length of 8-22 cm.

4. The drainage device of claim 1, wherein the one or more removable fixation systems are selected from the group consisting of flanges, balloons, foams, stickers, blocks, bumpers, tubes, rubber liners, and any combinations thereof.

5. The drainage device of claim 4, wherein the one or more fixation systems are one or more inflatable balloons.

6. The drainage device of claim 5, wherein the one or more inflatable balloons are inflatable with air, liquid or granular media.

7. The drainage device of claim 5, wherein the one or more inflatable balloons are inflatable to a diameter that is equal to or greater than a maximum diameter of the catheter.

8. The drainage device of claim 5, wherein the one or more inflatable balloons have a diameter of about 0.5-2.5 cm when inflated.

9. The drainage device of claim 5, wherein the one or more fixation systems further comprise one or more movable bumpers encircling a proximal portion of the catheter.

10. The drainage device of claim 5, wherein the one or more inflatable balloons are connected to and configured to be inflated by one or more inflation channels that extend along the longitudinal axis of the catheter.

11. The drainage device of claim 10, wherein the one or more inflation channels are arranged on an outer surface of the catheter.

12. The drainage device of claim 10, wherein the one or more inflation channels are arranged on an inner surface of the catheter.

13. The drainage device of claim 10, further comprising a clip for attaching the one or more inflation channels to the catheter.

14. The drainage device of claim 1, further comprising one or more one-way valves at the proximal end of the catheter.

15. The drainage device of claim 1, further comprising a pump, a syringe, an inflator bulb or an aspirator that is fluidly connected to the catheter to draw or drain the air or fluid from the body cavity.

16. The drainage device of claim 1, wherein the catheter further comprises antifibrinolytic, antithrombotic, anticoagulant, antibiotic or antiviral agents or any combinations thereof coating an inner surface of the catheter.

17. The drainage device of claim 1, wherein the device further comprises one or more luer-lock adapters at the proximal end of the catheter.

18. The drainage device of claim 1, further comprising 3-5 asymmetric holes on the distal end of the catheter for air release.

19. The drainage device of claim 1, further comprising a sensor for measuring and displaying a pressure in the body cavity.

20. A method for removing air or fluid from a body cavity comprising:
draining air or fluid from a subject in need thereof using the device as claimed in claim 5 by
i) inserting the catheter and at least one of the one or more inflatable balloons into the body cavity of the subject;
ii) inflating the one or more balloons to a size sufficient to hold the inserted catheter in place;
iii) draining out air or fluid from the body cavity through the catheter;
iv) deflating the at least one of the one or more balloons that are inside the body; and
v) removing the catheter and the deflated balloons as a single unit.

21. The method of claim 20, wherein at least one of the one or more balloons are inflated outside the body of the subject.

22. The method of claim 21, wherein the at least one or more balloons outside the body of the subject are inflated after the one or more balloons are inflated inside the body of the subject.

23. The method of claim 20, further comprising a step of sliding a bumper outside of the body cavity towards the distal end of the catheter after insertion to secure the catheter in place.

24. A method for treating a pneumothorax comprising:
draining air or fluid from a subject in need thereof by
i) inserting a needle and a catheter as a single unit through a chest wall of the subject so that a tip of the needle extends into the pleural cavity of the subject;
ii) inflating one or more balloons attached to a surface of the catheter to an appropriate size, wherein the size of the inflated balloon is sufficient to fix the catheter in position;
iii) retracting the needle with a retractable needle system and/or removing the needle;
iv) draining out the air or fluid from the pleural cavity through the catheter;
v) deflating the one or more balloons; and
vi) removing the catheter and the deflated balloons as a single unit.
